(19) United States
(12) Patent Application Publication
(10) Pub. No.: US 2005/0165428 A1
(43) Pub. Date: Jul. 28, 2005

(54) Absorable surgical structure

(76) Inventor: Kenneth L. Franco, Peoria, IL (US)

Correspondence Address: GIFFORD, KRASS, GROH, SPRINKLE & CITKOWSKI, P.C, PO BOX 7021, TROY, MI, 48007-7021, US

(21) Appl. No.: 11/066,928

(22) Filed: Feb. 25, 2005

Related U.S. Application Data
(63) Continuation-in-part of application No. 10/838,954, filed on May 4, 2004.
Application No. 10/838,954 is a continuation of application No. 09/966,800, filed on Sep. 25, 2001, now abandoned.
(60) Provisional application No. 60/259,997, filed on Jan. 5, 2001.
(60) Provisional application No. 60/235,036, filed on Sep. 25, 2000.

(51) Int. Cl. A61B 17/08

(52) U.S. Cl. 606/153

(57) ABSTRACT

A surgical structure includes a solid, biologically compatible material that dissolves in a physiologically functioning vessel within a few hours or days. A surgical structure as detailed herein facilitates the joinder of vessels with tissue adhesives and other surgically acceptable closures and is formed of solid physiologic saline, block copolymers such as polyethylene glycol, polysaccharides, polymerized proteins such as collagen or gelatin, and synthetic polymers such as poly lactic acid or poly glycolic acid. A method of sealing a vessel includes inserting a solid biologically compatible material surgical structure having an extending portion and an intersecting portion having two arms into an aperture within a vessel such that the extending portion of the surgical structure has a fluid opening extending from the vessel aperture. The structure dissolves within the vessel within a few hours or days. A blunt end cut conduit is placed over the extending portion until the conduit end contacts the vessel proximal to the vessel aperture. With the introduction of a tissue sealant or other closure to the region of contact between the blunt end cut of the conduit and the vessel, the conduit and vessel are adhesively joined together.

### RELATED APPLICATIONS

This application is a continuation-in-part of U.S. Utility application Ser. No. 10/838,954 filed May 4, 2004, which is a continuation of U.S. Utility application Ser. No. 09/966,800 filed Sep. 25, 2001, which in turn claims priority of U.S. Provisional Application No. 60/235,036 filed Sep. 25, 2000 and U.S. Provisional Application No. 60/259,997 filed Jan. 5, 2001.

### FIELD OF THE INVENTION

The present invention relates to the anastomosis of vessels or tissues, and more particularly to stents composed of biocompatible material designed to dissolve in the bloodstream within several hours to days, and plugs which also dissolve after being used to close an opening in any vessel or tissue.

### BACKGROUND OF THE INVENTION

Coronary artery disease is a major health problem with over 15,000,000 sufferers within the United States and about 500,000 additional cases each year. Symptomatic sufferers undergo percutaneous transluminal coronary angioplasty (PTCA)/stent or coronary artery bypass grafting (CABG). PTCA/stent as a percutaneous procedure is less invasive than open heart surgery but is limited in effectiveness owing to arterial stent re-stenosis. The alternative procedure, CABG performed with cardiopulmonary bypass or off-pump variants, requires an invasive incision including a median sternotomy in the case of complete revascularization to bypass all three major coronary arteries.

Owing to the limitations of existing surgical interventions, there is a need to develop a closed chest, totally endoscopic coronary artery bypass grafting procedure which is performed through a series of small chest incisions to access the coronary arteries. As part of such endoscopic procedure, there exists a need to connect the aorta or a coronary artery with a bypass conduit with an absorbable stent and sealing the anastomosis with a tissue sealant.

Present day surgical procedures in addition to coronary artery bypass grafting would benefit from the use of an absorbable stent joining vessels and sealing the resulting joint with a tissue sealant. In addition to coronary arteries, any arterial or venous anastomosis, vas deferens, and fallopian tubes may benefit from such a stent.

Whereas traditional sutures and staples cinch together tissue to form a closure, a tissue adhesive allows for a tissue closure to retain the natural tissue orientation. Without adequate mechanical support around an opening in any tissue, the full advantages of tissue adhesives are not obtained. Thus, there exists a need for a plug capable of providing support to the area around an opening in tissue to facilitate tissue adhesive closure.

Stents aid in holding vessel ends in a desired orientation during a surgical procedure and while vessel tissue fuses during healing. Typical absorbable stents have been made from biological materials which slowly are absorbed by body tissue in the course of healing. Stent biological materials are usually polymers which dissolve slowly over a period of weeks. U.S. Pat. Nos. 3,620,218; 3,683,926; 5,489,297; 5,653,744; and 5,762,625 are representative of conventional reabsorbable stent materials. Owing to the relatively slow reabsorption of prior art stents, the applications for absorbable stents have been limited.

U.S. Pat. No. 4,690,684 discloses frozen blood plasma stents which are cylindrical masses lacking a fluid communicating bore; these stents are operative in end-to-end vessel thermal bonding. These stents suffer the limitations as to sterility and usage only to those procedures requiring end-to-end vessel joinder. Thus, there exists a need for a sterile, reabsorbable stent capable of dissolution in the bloodstream in less than a few hours to days that is useful in cardiac bypass procedures and other procedures requiring a vascular anastomosis.

### SUMMARY OF THE INVENTION

A surgical structure includes a solid, biologically compatible material that dissolves in a physiologically functioning vessel within a few hours to days. The material is formed having an extending portion adapted to be received within a lumen of a vascular conduit. A surgical structure as detailed herein facilitates the joinder of vessels with tissue adhesives and other surgically acceptable closures. A surgical structure formed of solid physiologic saline, modified polyethylene glycol or other polymers with the surgical structure intermediate therein. A method of anastomosis includes inserting a solid biologically compatible material surgical structure having an extending portion and an intersecting portion having two arms into an aperture within a vessel such that the extending portion of the surgical structure has a fluid opening extending from the vessel aperture. The structure dissolves within the vessel within a few hours. A blunt end cut conduit is placed over the extending portion until the conduit end contacts the vessel proximal to the vessel aperture. With the introduction of a tissue sealant or other closure to the region of contact between the blunt end cut of the conduit and the vessel, the conduit and vessel are adhesively joined together.

A tissue plug is detailed that is formed of a biologically reabsorbable material cast to define a platen surface having a lateral dimension adapted to underlie a tissue opening. A tissue flap or other sealing skin is placed over the opening with pressure applied thereto being supported by the platen surface during a sealing process. A method for sealing such a tissue opening includes inserting a solid reabsorbable plug having a platen surface into a tissue opening such that the tissue opening contacts the platen surface. Adhering contacting portions of the tissue opening or overlying a separate sheet of material serves to form a closure.

### BRIEF DESCRIPTION OF THE DRAWINGS

FIG. 1(A) is an angled Y-letter shaped stent, (B) is a partial Y-letter shaped stent, (C) is a T-shaped stent, and (D) is a cylindrical stent.

FIG. 2 is a schematic drawing illustrating the steps for conducting an anastomosis according to the present invention, 2(A) shows an incised vessel separated from a stent according to the present invention and a bypass conduit, 2(B) shows the insertion of a stent according to the present invention into the incised vessel, 2(C) shows the insertion of a stent portion into the bypass conduit, and 2(D) shows the completed anastomosis of the vessel and bypass conduit with tissue sealant.

FIGS. 3 (A)-(D), collectively referred to as FIG. 3, illustrate various plugs according to the present invention.

### DETAILED DESCRIPTION OF THE PREFERRED EMBODIMENTS

An absorbable stent or plug according to the present invention has utility in combination with a tissue adhesive in an anastomosis procedure or as a substitute for conventional closures of tissue openings such as sutures and staples. An absorbable stent or plug according to the present invention is composed of a sterile, biologically compatible substance capable of dissolution within the human body in less than a few hours to days. According to the present invention, dissolution in the body typically occurs within a month. It is appreciated that certain uses dictate a dissolution rate of less than one week and even within one hour. The dissolution time of an inventive stent or plug is controlled by factors including melting temperature, enzymatic degradation rate, article thickness, and coatings. A stent or plug according to the present invention is composed of modified block copolymers such as polyethylene glycol; frozen super cooled physiological saline; polysaccharides such as pectin; proteinaceous polymers such as albumin, fibrin and collagen; gelatin; or synthetic polymers such as poly lactic acid or poly glycolic acid; and combinations thereof. By way of example, a base structure of an inventive stent or plug is formed of a first substance such as a PEG wax and coated with an albumin coating to modify hydrophilicity and the dissolution rate of the resulting stent or plug. Typical coating thicknesses range from 0.1 to 1200 microns. Optionally, additives can be incorporated into an absorbable stent or plug prior to development or freezing. Optional additives illustratively include an elasticizer such as glycerol, an anti-coagulant such as heparin, a biocompatible colorant or other substances. Preferably, an inventive surgical structure dissolves fairly rapidly and completely to preclude an embolism associated with a free-floating structure remnant.

A stent or plug according to the present invention is produced by pouring a sterile stent solution into a sterile mold cavity to solidify through polymerization or cooling the stent solution until solid, the mold cavity being composed of stainless steel, elastomeric or thermoplastic tubing, glass or other substances. A stent according to the present invention is preferably cast with hollow channels therethrough, but the plug is solid. Optionally, a stent according to the present invention is cast solid and bored to produce a hollow communication passage therethrough. A saline stent or plug according to the present invention is frozen through placement in a cryofreezer containing a stable temperature below about −40° C. A stent or plug is solidified through immersion or thermal contact with a liquid nitrogen bath or left to harden as a wax. A proteinaceous stent or plug is polymerized with a chemical cross-linker such as an aldehyde or thermally polymerized. Thermal polymerization is preferable in transient applications in the event that thermally sensitive wax or saline is present. A stent or plug according to the present invention upon removal from the mold possesses a hard, glassy or wax-like quality.

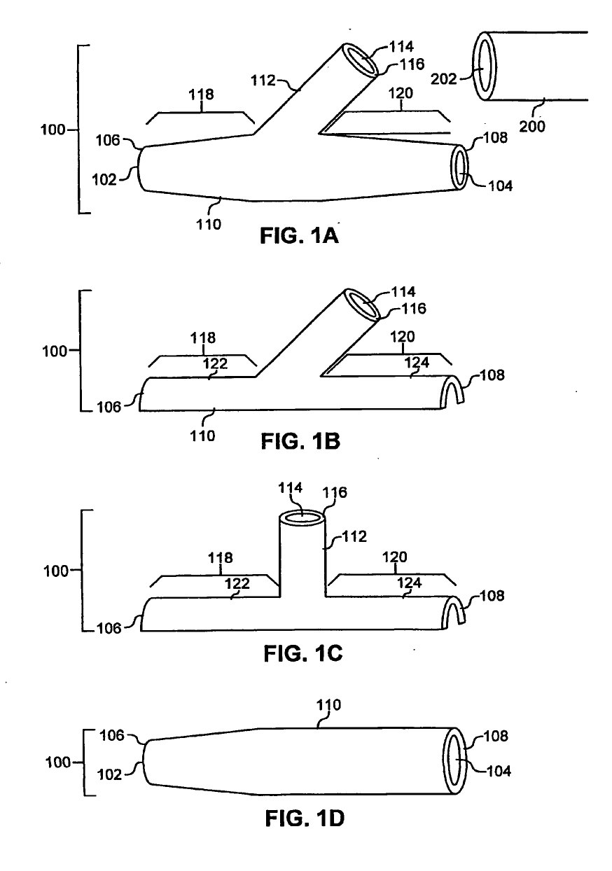

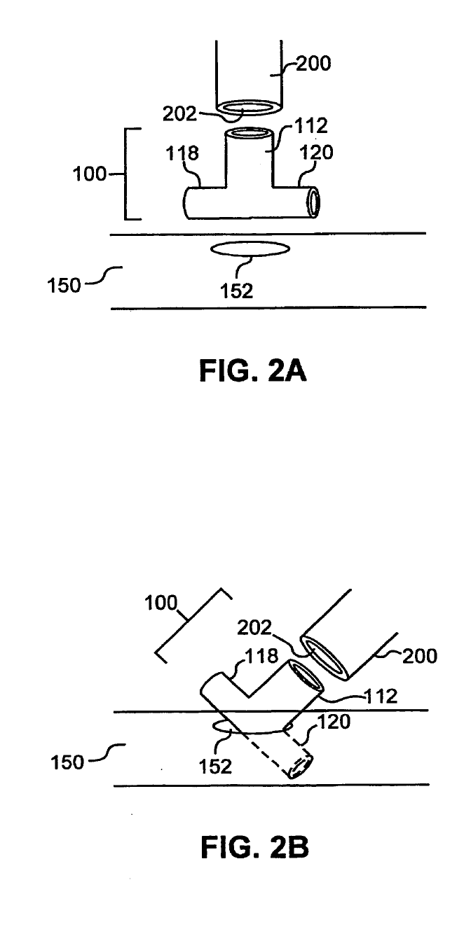

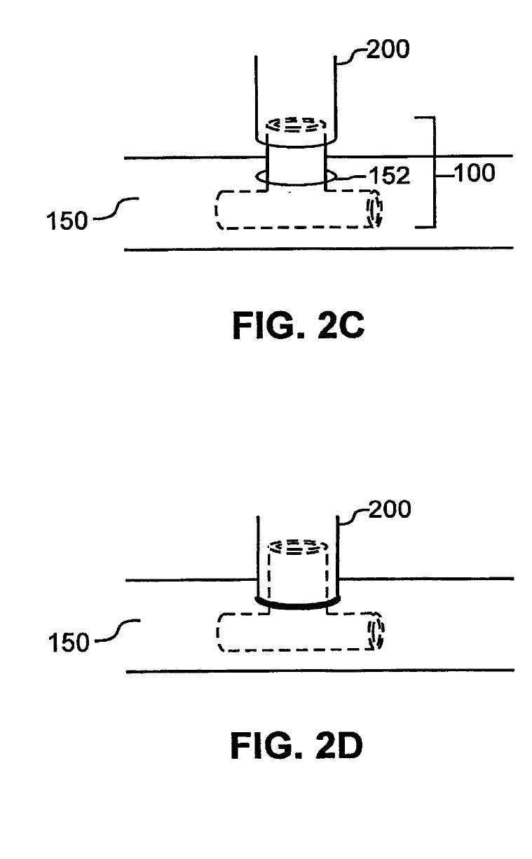

Referring now to FIG. 1, a stent according to the present invention is shown generally at 100, with like features being like numbered. Those stents depicted in FIGS. 1(A)-(C) each have a cylindrical portion 112 adapted to insert within a severed vessel, biological, or synthetic bypass conduit shown at 200 and defining a lumen 202. In the stents depicted in FIGS. 1(A)-(C), an intersecting portion 116 is adapted to insert through an incision within a blood vessel such as that depicted at 152 in FIG. 2. Preferably, the incision dimensions are complementary to the dimensions of the lumen 202. The intersecting portion 114 has two arms 118 and 120 that define primary portion 110. While it is appreciated that the dimensions of each arm 118 and 120, and the cylindrical portion 112, are readily formed to engage a variety of vessel and/or conduit sizes, typical dimensions for a stent according to the present invention operative in a coronary artery bypass are about 1 to 1½ centimeters for arm 120 and ½ to ¾ centimeters for arm 118, independently, with an external diameter of about 1 to about 4 millimeters, and a cylindrical portion 112 having a length of about 1½ to 2½ centimeters in length and having an outer diameter of from about 1 to about 8 millimeters. Preferably, the ends of the arms 118 and 120 taper to a smaller external diameter termini 106 and 108 to facilitate insertion. An optional fluid communicative channel is defined between at least two of the openings 102, 104 and 114.

In another embodiment of the present invention shown in FIG. 1(B), a non-circumferential Y stent is provided that causes less obstruction of a vessel during insertion as compared to the embodiment of the present invention depicted in FIG. 1(A). The stent of FIG. 1(B) has surfaces 122 and 124 associated with arms 118 and 120, respectively, that generally conform to the interior curvature of the vessel 150 depicted in FIG. 2.

FIG. 1(C) shows a T-letter shaped non-circumferential absorbable stent according to the present invention. A T-letter shaped stent according to the present invention is contemplated to be particularly well suited for an aortic anastomosis procedure. A T-letter shaped stent has a cylindrical portion 112 extending in a generally orthogonal direction away from a vessel intersecting portion arms 118 and 120. Preferably, the termini 116, 106 and 108 of the extending portion 112, and the extending arms 118 and 120, respectively, are tapered to facilitate insertion within a bypass conduit or vessel 150 as depicted in FIG. 2. Optionally an opening 114 in the terminus 116 provides fluid communication between interior surfaces of the stent. An aortic absorbable stent according to the present invention typically has arms 120 having a length of from about 1 l₂ to 2 centimeters and 118 arm having a length of ½ to 1 centimeter and an outer diameter of from about 8 millimeters to 11 millimeters, with the cylindrical portion 112 having a length of from about 1½ to 2½ centimeters in length and an outer diameter of between 1 and 8 millimeters. Preferably, the cylindrical portion 112 has a length greater than an extending arm 118 or 120.

In another embodiment of the present invention, an absorbable stent is utilized to form an end-to-end joint between two vessels, as provided for in FIG. 1(D). The cylindrical stent 100 in FIG. 1(D) has a primary portion 110 with an overall length of about 2 to 3½ centimeters and an external diameter adapted to insert within a vessel to be joined. It is appreciated that the external diameter of the stent 100 in FIG. 1(D) need not be uniform along the length. A varying external diameter stent 100 in FIG. 1(D) is well suited for engaging two vessels of varying lumenal dimensions. Preferably, the termini 106 and 108 taper to promote insertion within a vessel. An optional fluid communication channel is provided between openings 102 and 104.

Typical of embodiments according to the present invention, the internal fluid communicating channel has a diameter of 1 to 6 millimeters.

FIG. 2 illustrates the steps for performing an anastomosis according to the present invention. FIG. 2(A) shows a blood vessel 150 having a sidewall aperture therein 150 to be connected to a blunt end cut vessel or conduit 200 by way of a stent 100 as shown in FIG. 1(C). In FIG. 2(B), an arm 120 is inserted through the aperture 152 in the vessel 150 with an angular motion relative to the walls of vessel 150. The stent 100 is then pulled against the vessel sidewalls defining the aperture 152 until extending arm 118 also enters the vessel 150. FIG. 2(C) shows the engagement of lumen 202 of blunt end cut conduit 200 with the extending portion 112 of the stent 100. Conduit 200 is slipped over extending portion 112 towards the vessel 150. Excessive blood and moisture are optionally removed from the region around the aperture 152 and a tissue adhesive is applied about the aperture 152 and/or the end of conduit 200 as the conduit 200 is brought into physical contact with the vessel 150. The tissue sealant includes new tissue adhesives developed which are as strong as materials including proteinaceous adhesives such as those including albumin and fibrin; and alkyl cyano acrylate monomers. After the tissue adhesive is in simultaneous contact with the vessel 150 and conduit 200 for three minutes, polymerization is complete, as shown in FIG. 2(D). With the rapid dissolution of a stent according to the present invention, the integrity of the resulting tissue adhesive joint is readily monitored during the course of the surgical procedure thereby allowing for correction of seepage from the resultant joint.

In another embodiment of the present invention, a dissolvable plug is provided to cover an opening in a vessel or tissue to facilitate the use of a tissue sealant to close the opening.

As used herein “opening” is defined to mean any cut, tear, laceration or fissure in any living tissue.

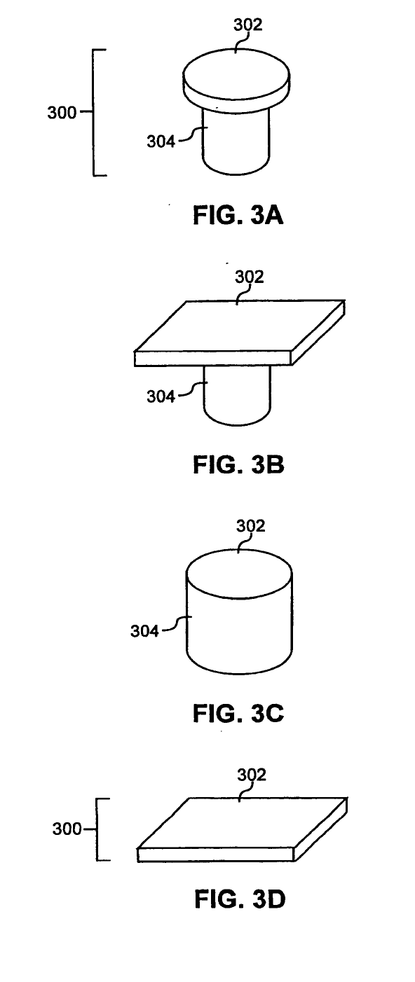

A closing plug according to the present invention is shown generally at 300 in FIGS. 3(A)-(C). The plug 300 has a platen surface 302 and a supporting structure 304. The plug 300 is formed of any reabsorbable material under conditions found within a living organism. These materials illustratingly include frozen saline, block copolymers such as PEG, frozen blood plasma, collagen, gelatin, and polysaccharides, proteinaceous polymers as detailed herein, and reabsorbable materials known to the art including synthetic polytmers such as poly lactic acid and poly glycolic acid. Reabsorption times range from between a few hours or days. While the embodiment of the plug 300 depicts a supporting portion 304 as being generally columnar in shape, it is appreciated that a variety of support structure shapes are operative herein.

In operation, a closing plug according to the present invention is inserted through an opening in a tissue and affords a mechanical base which seals the opening and facilitates application of the tissue sealant. It is appreciated that the relative size and shape of the platen relative to the base portion of a plug is variable to accommodate tissue adhesive closing of openings within a variety of tissues. A closing plug according to the present invention is operative in sealing openings in tissues illustratively including blood vessels, intestines, the stomach, fluid ducts including hepatic, bile, tear, cranial, seminal and the like.

In the instance where a large opening in tissue is being closed, a planar or a substratum-conforming platen 300 as shown in FIG. 3(D) is laid over the opening prior to applying tissue sealant. A tissue flap closure plug 300 as shown in FIG. 3(D) thus functions independent of a pedestal portion.

The patents cited herein are indicative of the level of skill in the art to which the invention pertains. Each patent is hereby incorporated by reference to the same extent as if each individual patent was specifically and individually incorporated herein by reference.

The present invention has been described in detail for the purpose of illustration, and it is to be understood that such detail is solely for that purpose and that variations will be readily apparent to those skilled in the art without departing from the spirit and scope of the invention as defined by the appended claims.

1. A surgical structure comprising a solid, biologically compatible material dissolving in a physiologically functioning vessel within a few hours or days, said solid biological material formed having an extending portion adapted to be received within the lumen of a vascular conduit.

2. The structure of claim 1 further comprising an intersecting portion having two arms, the intersecting portion adapted to be received within a vascular aperture.

3. The structure of claim 1 wherein said solid biologically compatible material is selected from the group consisting of: frozen physiologic saline, block copolymers such as polyethylene glycol, polysaccharide, polymerized protein including collagen and gelatin, and synthetic polymers such as poly lactic acid and poly glycolic acid

4. The structure of claim 1 further comprising a biodegradable coating on said biologically compatible material.

5. The structure of claim 2 wherein the arms of the intersecting portion have tapered ends of unequal length.

6. The structure of claim 1 wherein the extending portion is angled to the intersecting portion and the vessel is a coronary artery.

7. The structure of claim 6 wherein the arms each have a length of about 0.5 to 2 centimeters and a diameter of about 1 to 4 millimeters, and the extending portion has a length of about 1.5 to 2.5 centimeters and a diameter of about 0.1 to 0.8 centimeters.

8. The structure of claim 2 wherein the extending portion is approximately orthogonal to the intersecting portion and the vessel is an aorta.

9. The structure of claim 6 wherein the arms each have a length of about 1.5 to 2 centimeters and a diameter of about 10 millimeters, and the extending portion has a length of about 1.5 to 2.5 centimeters and a diameter of about 0.1 to 0.8 centimeters.

10. A surgical structure formed from a biologically compatible and dissolvable material selected from the group consisting of: saline, block copolymers such as polyethylene glycol, polysaccharide, polymerized proteins such as collagen and gelatin, and synthetic polymers such as poly lactic acid and poly glycolic acid; and having a fluid communicating bore therein.

11. A method of anastomosis comprising:
inserting a solid biologically compatible surgical structure of claim 2 into an aperture within a vessel such that the extending portion of said structure having a fluid opening extends from the vessel aperture, said structure dissolving within said vessel within a few hours or days;
receiving a blunt end cut conduit over the extending portion until the conduit end contacts the vessel about the vessel aperture; and
introducing a tissue sealant to a region of contact between the blunt end of the conduit and the vessel.

12. A tissue plug comprising a biologically reabsorbable material cast to define a platen surface having a lateral dimension adapted to underlie a tissue opening intended to be sealed.

13. The plug of claim 12 wherein the platen surface is supported by a pedestal structure having a pedestal lateral dimension.

14. The plug of claim 13 wherein the platen surface lateral dimension is equal to the pedestal structure lateral dimension.

15. The plug of claim 13 wherein the platen surface lateral dimension is greater than the pedestal structure lateral dimension.

16. The plug of claim 12 wherein the platen surface is nonplanar.

17. The plug of claim 12 wherein said biological reabsorbable material is selected from the group consisting of: saline, block copolymers such as polyethylene glycol, blood plasma, polysaccharides, polymerized proteins such as collagen and gelatin, and synthetic polymers such as poly lactic acid and poly glycolic acid.

18. A method of sealing a tissue opening comprising:
inserting a solid reabsorbable plug having a platen surface with a lateral dimension interior to the tissue opening such that the tissue opening contacts the platen surface; and
adhering contacting portions of the tissue opening to form a closure.

19. The method of claim 18 wherein adhering the tissue opening is accomplished with a tissue sealant.

20. The method of claim 18 wherein the tissue opening is located in a tissue selected from the group consisting of blood vessel, intestine, and biological fluid duct.
